(19) United States
(12) Patent Application Publication
(10) Pub. No.: US 2024/0122973 A1
(43) Pub. Date: Apr. 18, 2024

(54) LIGAND-BOUND ZINC SULFIDE NANOPARTICLES, METHODS FOR MAKING THE SAME, AND THEIR USE FOR TREATMENT

(71) Applicant: Wuhan Vast Conduct Science Foundation CO., LTD., WUHAN (CN)

(72) Inventors: Taolei SUN, WUHAN (CN); Guanbin GAO, WUHAN (CN); Meng YU, WUHAN (CN)

(73) Assignee: Wuhan Vast Conduct Science Foundation CO., LTD., WUHAN (CN)

(21) Appl. No.: 18/546,270

(22) PCT Filed: Mar. 23, 2021
(86) PCT No.: PCT/CN2021/082357

§ 371 Date: Aug. 12, 2023

(51) Int. Cl. A61K 33/30; A61P 25/28; A61P 29/00; B82Y 5/00; B82Y 40/00

(52) U.S. Cl. CPC A61K 33/30; A61P 25/28; A61P 29/00; B82Y 5/00; B82Y 40/00

(57) ABSTRACT

Ligand-bound zinc sulfide nanoparticles, the method of preparing the ligand-bound zinc sulfide nanoparticles, composition containing ligand-bound zinc sulfide nanoparticles, uses of the ligand-bound zinc sulfide nanoparticles and composition containing the ligand-bound zinc sulfide nanoparticles, including inhibiting the fibrosis of amyloid-β (Aβ) and reducing the expression of inflammatory factors, treatment of Aβ fibrosis-caused/related Alzheimer's disease (AD), cerebral amyloid angiopathy (CAA), retinal ganglion cell degeneration in glaucoma (RGCD) or myositis/myopathy (MM), and to prepare drugs for the treatment of AD, CAA, RGCD or MM, and methods for treating the above diseases.

### FIELD OF THE INVENTION

The present invention relates to the technical field of nanotechnology and its applications, particularly to ligand-bound zinc sulfide nanoparticles, composition comprising the ligand-bound zinc sulfide nanoparticles, use of the ligand-bound zinc sulfide nanoparticles to prepare medications for treatment, and methods employing the ligand-bound zinc sulfide nanoparticles and composition for treatment.

### BACKGROUND OF THE INVENTION

Amyloid-β (Aβ) fibrosis causes or is associated with diseases, such as Alzheimer's disease (AD), cerebral amyloid angiopathy (CAA), retinal ganglion cell degeneration in glaucoma (RGCD), myositis/myopathy (MM).

Alzheimer's disease (AD) is a chronic neurodegenerative disease. Its pathological features include extracellular senile plaque deposition, intracellular neurofibrillary tangles and abnormal loss of neurons and synapses. More and more evidences show that the deposition of extracellular senile plaques and the initial accumulation of intracellular neurofibrillary tangles can trigger a series of serious pathological processes, including human astrocyte (HA) proliferative inflammation and oxidative stress. HA proliferative inflammation and oxidative stress can accelerate the accumulation of senile plaques and neurofibrillary tangles. This vicious cycle can lead to abnormal neuronal and synaptic loss. The deposition of senile plaques is caused by misfolding, abnormal aggregation and fibrosis of Aβ. Therefore, it is of great significance to develop potential drugs that can simultaneously reduce Aβ plaque and neuroinflammation.

### SUMMARY OF THE INVENTION

The present invention provides ligand-bound zinc sulfide nanoparticle (R-ZnS NPs). In certain embodiments, the ligand-bound zinc sulfide nanoparticle comprises a zinc sulfide core; and a ligand (R) bound to the zinc sulfide core. In certain embodiments, the ligand (R) is one selected from the group consisting of L-cysteine, D-cysteine, N-isobutyryl-L-cysteine (L-NIBC), N-isobutyrul-D-cysteine (D-NIBC), N-acetyl-L-cysteine (L-NAC), and N-acetyl-D-cysteine (D-NAC). In certain embodiments, the ligand (R) is captopril. In certain embodiments of the ligand-bound zinc sulfide nanoparticle, the diameter of the zinc sulfide core is 0.5-4.0 nm. In certain embodiments of the ligand-bound zinc sulfide nanoparticle, the diameter of the zinc sulfide core is 1.0-3.5 nm.

The present invention provides a process of preparing ligand-bound zinc sulfide nanoparticles (R-ZnS NPs). In certain embodiments, the process comprises:
dissolving a ligand (R) in deionized water, resulting in a ligand aqueous solution; wherein the concentration of ligand in the ligand aqueous solution is 0.02-2.0 mol/L;
adding zinc acetate solution into the ligand aqueous solution, resulting in a zinc acetate/ligand reaction mixture; wherein the concentration of the zinc acetate aqueous solution is 0.01-1.0 mol/L; and wherein the molar ratio of ligand to zinc acetate ranges from 1:1 to 10:1;
adjusting the pH of the zinc acetate/ligand reaction mixture to the range of 7-10;
adding sodium sulfide aqueous solution dropwise into the pH-adjusted zinc acetate/ligand reaction mixture, resulting in a sodium sulfide/zinc acetate/ligand reaction mixture; wherein the molar ratio of the added sodium sulfide to the zinc acetate in the zinc acetate/ligand reaction mixture ranges from 0.1:1-5:1;
heating the sodium sulfide/zinc acetate/ligand reaction mixture to a predetermined temperature, and the reaction is maintained for a predetermined time, resulting in the formation of R-ZnS NPs; wherein the predetermined temperature is 50 - 100 degrees Celsius; and wherein the predetermined time is 1-5 hours.

In certain embodiments, the process further comprises purifying the R-ZnS NPs by centrifugation with an ultrafiltration tube; wherein the ultrafiltration tube is with a molecular weight cut-off of 5k Daltons.

The present invention provides ligand-bound zinc sulfide nanoparticles for use in treatment of a subject with Alzheimer's disease (AD) and cerebral amyloid angiopathy (CAA), retinal ganglion cell degeneration in glaucoma (RGCD) or myositis/myopathy (MM). In certain embodiments, the ligand of the ligand-bound zinc sulfide nanoparticles is one selected from the group consisting of L-cysteine, D-cysteine, N-isobutyryl-L-cysteine (L-NIBC), N-isobutyrul-D-cysteine (D-NIBC), N-acetyl-L-cysteine (L-NAC), and N-acetyl-D-cysteine (D-NAC). In certain embodiments, the ligand of the ligand-bound zinc sulfide nanoparticles is captopril.

The present invention provides a composition comprising ligand-bound zinc sulfide nanoparticles, where the composition is used for treatment of a subject with Alzheimer's disease (AD) and cerebral amyloid angiopathy (CAA), retinal ganglion cell degeneration in glaucoma (RGCD) or myositis/myopathy (MM). In certain embodiments, the ligand of the ligand-bound zinc sulfide nanoparticles is one selected from the group consisting of L-cysteine, D-cysteine, N-isobutyryl-L-cysteine (L-NIBC), N-isobutyrul-D-cysteine (D-NIBC), N-acetyl-L-cysteine (L-NAC), and N-acetyl-D-cysteine (D-NAC). In certain embodiments, the ligand of the ligand-bound zinc sulfide nanoparticles is captopril.

The present invention provides ligand-bound zinc sulfide nanoparticles for use in treatment of a subject with a condition of excessive expression of interleukin-6 (IL-6), interleukin-8 (IL-8), interleukin-1β (IL-1β), hypersensitive-c-reactive-protein (Hs CRP), or tumor necrosis factor-alpha (TNFα). In certain embodiments, the ligand of the ligand-bound zinc sulfide nanoparticles is one selected from the group consisting of L-cysteine, D-cysteine, N-isobutyryl-L-cysteine (L-NIBC), N-isobutyrul-D-cysteine (D-NIBC), N-acetyl-L-cysteine (L-NAC), and N-acetyl-D-cysteine (D-NAC). In certain embodiments, the ligand of the ligand-bound zinc sulfide nanoparticles is captopril.

The present invention provides a composition comprising ligand-bound zinc sulfide nanoparticles, where the composition is used for treatment of a subject with a condition of excessive expression of interleukin-6 (IL-6), interleukin-8 (IL-8), interleukin-1β (IL-1β), hypersensitive-c-reactive-protein (Hs CRP), or tumor necrosis factor-alpha (TNFα). In certain embodiments, the ligand of the ligand-bound zinc sulfide nanoparticles is one selected from the group consisting of L-cysteine, D-cysteine, N-isobutyryl-L-cysteine (L-NIBC), N-isobutyrul-D-cysteine (D-NIBC), N-acetyl-L-cysteine (L-NAC), and N-acetyl-D-cysteine (D-NAC). In certain embodiments, the ligand of the ligand-bound zinc sulfide nanoparticles is captopril.

The objectives and advantages of the invention will become apparent from the following detailed description of preferred embodiments thereof in connection with the accompanying drawings.

### DESCRIPTION OF THE DRAWINGS

Preferred embodiments according to the present invention will now be described with reference to the Figures, in which like reference numerals denote like elements.

FIG. 1 shows the TEM image and curves of a group of physical characteristics of Cap-ZnS NPs: (A) TEM image of Cap-ZnS NPs; (B) statistical chart of particle size and size distribution of Cap-ZnS NPs; (C) infrared spectrum of Cap-ZnS NPs; (D) X-ray photoelectron spectrum of Cap-ZnS NPs; (E) Zn spectrum of Cap-ZnS NPs and (F) S spectrum of Cap-ZnS NPs.

FIG. 2 shows the ThT kinetics curves of Cap-ZnS NPs, MA-ZnS NPs and DHLA-ZnS NPs incubated with Aβ₄₀ for 60 hours respectively, illustrating the effects of different concentrations of (A) Cap-ZnS NPs, (B) MA-ZnS NPs and (C) DHLA-ZnS NPs on the fibrosis kinetics of Aβ₄₀ at 20 μM.

FIG. 3 shows the AFM and TEM images of Cap-ZnS NPs incubated with Aβ₄₀ for 60 hours: (A), (B), (C) show the AFM images when the final concentration of Cap-ZnS NPs is 0, 1 and 5 ppm respectively; (D), (E), (F) show the TEM images when the final concentration of Cap-ZnS NPs is 0, 1 and 5 ppm respectively.

FIG. 4 is a histogram of the survival rates of PC12 cells, showing (A) the effects of different concentrations of Cap-ZnS NPs on the survival rates of PC12 cells; (B) the inhibitory effects of different concentrations of Cap-ZnS NPs on the cytotoxicity of Aβ₄₀ (final concentration of 25 pM).

FIG. 5 is a bar chart of the effect of Cap-ZnS NPs and Cap on the expression of five inflammatory factors in LPS model detected by ELISA: (A) IL-6, (B) IL-8, (C) IL-1 β, (D) hs CRP and (E) TNF-α.

FIG. 6 shows the HE staining images of the tissue sections of heart, liver, spleen, lung, kidney and brain of mice after intraperitoneal injection of 100 mg/kg Cap-ZnS NPs.

FIG. 7 shows the distribution of Cap-ZnS NPs in heart, liver, spleen, lung, kidney and brain at 2, 6, 12 and 24 hours after intraperitoneal injection of 20 mg/kg Cap-ZnS NPs (n=5).

FIG. 8 shows the results of Morris water maze in male mice after four weeks of daily administration of Cap-ZnS NPs, MA-ZnS NPs or DHLA-ZnS NPs: (A) latency period; (B) number of platform crossings; (C) swimming time in target quadrant; (D) stay time in target quadrant.

FIG. 9 shows the immunohistochemical images of Aβ₄₀, IL-1β, TNF-α and GFAP in the hippocampus: normal mice were the control group injected intraperitoneally with 20 mg/kg Cap-ZnS NPs; 60^{th} AD mice were the model control group at the 60^{th} week; and 64^{th} AD mice were the mice injected daily with 20 mg/kg Cap-ZnS NPs from the 60th week to the 64th week.

### DETAILED DESCRIPTION OF THE EMBODIMENTS

The present invention may be understood more readily by reference to the following detailed description of certain embodiments of the invention.

Throughout this application, where publications are referenced, the disclosures of these publications are hereby incorporated by reference, in their entireties, into this application in order to more fully describe the state of art to which this invention pertains.

As used herein, “administering” means oral (“po”) administration, administration as a suppository, topical contact, intravenous (“iv”), intraperitoneal (“ip”), intramuscular (“im”), intralesional, intrahippocampal, intracerebroventricular, intranasal or subcutaneous (“sc”) administration, or the implantation of a slow-release device e.g., a mini-osmotic pump or erodible implant, to a subject. Administration is by any route including parenteral and transmucosal (e.g., oral, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

The terms “systemic administration” and “systemically administered” refer to a method of administering a compound or composition to a mammal so that the compound or composition is delivered to sites in the body, including the targeted site of pharmaceutical action, via the circulatory system. Systemic administration includes, but is not limited to, oral, intranasal, rectal and parenteral (i.e. other than through the alimentary tract, such as intramuscular, intravenous, intra-arterial, transdermal and subcutaneous) administration, with the proviso that, as used herein, systemic administration does not include direct administration to the brain region by means other than via the circulatory system, such as intrathecal injection and intracranial administration.

As used herein, the terms “treating” and “treatment” refer to delaying the onset of, retarding or reversing the progress of, or alleviating or preventing either the disease or condition to which the term applies, or one or more symptoms of such disease or condition.

The terms “patient,” “subject” or “individual” interchangeably refers to a mammal, for example, a human or a non-human mammal, including primates (e.g., macaque, pan troglodyte, pongo), a domesticated mammal (e.g., felines, canines), an agricultural mammal (e.g., bovine, ovine, porcine, equine) and a laboratory mammal or rodent (e.g., rattus, murine, lagomorpha, hamster, guinea pig).

As used herein, the term “room temperature” means about 22-25 degree Celsius.

The present invention provides ligand-bound zinc sulfide nanoparticles (R-ZnS NPs).

In certain embodiments, the ligand-bound zinc sulfide nanoparticle (R-ZnS NPs) comprises a ligand (R) and a zinc sulfide core, wherein the ligand is bound to the zinc sulfide core. The ligand being bound to the zinc sulfide core means that the ligand forms a stable nanoparticle in solution with the zinc sulfide core through covalent bonds, hydrogen bonds, electrostatic force, hydrophobic force, van der Waals force, etc. In certain embodiments, the zinc sulfide core has a diameter of 0.5-4.0 nanometers (nm). In certain embodiments, the diameter of the zinc sulfide core is in the range of 1.0-3.5 nm.

In certain embodiments, the ligand of the ligand-bound zinc sulfide nanoparticles is one selected from the group consisting of L-cysteine, D-cysteine, N-isobutyryl-L-cysteine (L-NIBC), N-isobutyrul-D-cysteine (D-NIBC), N-acetyl-L-cysteine (L-NAC), and N-acetyl-D-cysteine (D-NAC).

In certain embodiments, the ligand of the ligand-bound zinc sulfide nanoparticle is Captopril (i.e. 1-[(S)-3-Mercapto-2-methylpropionyl]-L-proline) that is represented by formula (I):

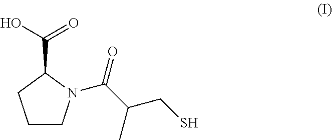

The present invention provides a process for preparing ligand-bound zinc sulfide nanoparticles (R-ZnS NP).

In certain embodiments, the process for preparing ligand-bound zinc sulfide nanoparticles (R-ZnS NP) comprises:
dissolving ligand in deionized water, resulting in a ligand aqueous solution; in certain embodiments, the concentration of ligand in the ligand aqueous solution is 0.02-2.0 mol/L, preferably 0.02-0.2 mol/L;
adding zinc acetate solution into the ligand aqueous solution, resulting in a zinc acetate/ligand reaction mixture; in certain embodiments, the zinc acetate/ligand reaction mixture was stirred at room temperature for 0.1-3 hours, preferably 0.3 -1.5 hours; in certain embodiments, the concentration of the zinc acetate aqueous solution is 0.01-1.0 mol/L, preferably 0.02-0.2 mol/L; in certain embodiments, the molar ratio of ligand to zinc acetate ranges from 1:1 to 10:1, preferably 1:1 to 5:1;
adjusting the pH of the zinc acetate/ligand reaction mixture to the range of 7-10, preferably 8-9; in certain embodiments, the pH-adjusted zinc acetate/ligand reaction mixture is stirred at room temperature for 0.3-5 hours, preferably 0.5-2 hours; in certain embodiments, the reagent used to adjust the pH is sodium hydroxide solution;
adding sodium sulfide aqueous solution dropwise into the pH-adjusted zinc acetate/ligand reaction mixture, resulting in a sodium sulfide/zinc acetate/ligand reaction mixture; in certain embodiments, the sodium sulfide/Zinc acetate/ligand reaction mixture is stirred at room temperature for 1-5 hours, preferably 1-3 hours; in certain embodiments, the molar ratio of the added sodium sulfide to the zinc acetate in the zinc acetate/ligand reaction mixture ranges from 0.1:1-5:1; preferably 0.2:1-2:1;
heating the sodium sulfide/zinc acetate/ligand reaction mixture to a predetermined temperature, and the reaction is maintained for a predetermined time, resulting in the formation of R-ZnS NPs; in certain embodiments, the predetermined temperature is 50-100 degrees Celsius, preferably 50-70 degrees Celsius; in certain embodiments, the predetermined time is 1-5 hours, preferably 1-2 hours.

In certain embodiments, the process further comprises:
purifying the R-ZnS NPs by centrifugation with an ultrafiltration tube; in certain embodiments, the conditions for centrifugation are 5000-6000 r/min, 5 minutes; in certain embodiments, the ultrafiltration tube is with a molecular weight cut-off of 5 k daltons;
collecting the liquid from the top portion of the ultrafiltration tube to obtain the purified R-ZnS NPs; in certain embodiments, the separated R-ZnS NPs are washed with ultrapure waters e.g. three times;
freeze-drying the purified L-ZnS NPs to obtain stable powder of L-ZnS NPs.

The present invention provides the ligand-bound zinc sulfide nanoparticles (R-ZnS NPs) for use in treatment of a subject with a condition of excessive expression of interleukin-6 (IL-6), interleukin-8 (IL-8), interleukin-10 (IL-1β), hypersensitive-c-reactive protein (Hs CRP), or tumor necrosis factor-alpha (TNFα). Here, the “excessive expression” means that the protein level is at least 20% higher than physiological expression level. The treatment is administration of the R-ZnS NPs or a composition containing the R-ZnS NPs. The treatment can decrease at least 50%, preferably 60%, 70%, 80%, 90% or 100%, of excess expressions of IL-6, IL-8, IL-10, Hs CRP, or TNFa, where the “excess expression” is defined as the difference between the expression level under the physiological condition and the expression level under the condition of excessive expression. In certain embodiments, the condition of excessive expression is induced by infection by microorganisms including fungi, bacteria and viruses. When a subject is infected by the pathological microorganisms, they secrete certain materials such as lipopolysaccharides (LPS) that will induce the excess expression of cytokines. LPS, also known as endotoxins, are large molecules consisting of a lipid and a polysaccharide found in the outer membrane of Gram-negative bacteria. Gram-negative bacteria are bacteria that do not retain the crystal violet stain used in the gram-staining method of bacterial differentiation. The gram-negative bacteria, including Escherichia coli (E. coli), Salmonella, Shigella, Pseudomonas, Moraxella, Helicobacter pylori, Stenotrophomonas, Bdellovibrio, acetic acid bacteria, Legionella, cyanobacteria, spirochaetes, green sulfur, green non-sulfur bacteria,Neisseria gonorrhoeae, Neisseria meningitidis, Moraxella catarrhalis, Haemophilus influenzae, Klebsiella pneumoniae, Legionella pneumophila, Pseudomonas aeruginosa, Proteus mirabilis, Enterobacter cloacae, Serratia marcescens, Salmonella enteritidis, Salmonella typhi, Acinetobacter baumannii. In certain embodiments, the condition of excessive expression is induced by autoimmune diseases or chronic inflammation disease including cancers. In certain embodiments, the ligand of the ligand-bound zinc sulfide nanoparticles is one selected from the group consisting of L-cysteine, D-cysteine, N-isobutyryl-L-cysteine (L-NIBC), N-isobutyrul-D-cysteine (D-NIBC), N-acetyl-L-cysteine (L-NAC), and N-acetyl-D-cysteine (D-NAC). In certain embodiments, the ligand of the ligand-bound zinc sulfide nanoparticles is Captopril.

The present invention provides a composition comprising ligand-bound zinc sulfide nanoparticles, where the composition is used for treatment of a subject with a condition of excessive expression of interleukin-6 (IL-6), interleukin-8 (IL-8), interleukin-1β (IL-1β), hypersensitive-c-reactive-protein (Hs CRP), or tumor necrosis factor-alpha (TNFα). In certain embodiments, the ligand of the ligand-bound zinc sulfide nanoparticles is one selected from the group consisting of L-cysteine, D-cysteine, N-isobutyryl-L-cysteine (L-NIBC), N-isobutyrul-D-cysteine (D-NIBC), N-acetyl-L-cysteine (L-NAC), and N-acetyl-D-cysteine (D-NAC). In certain embodiments, the ligand of the ligand-bound zinc sulfide nanoparticles is Captopril.

The present invention provides a pharmaceutical composition for the treatment of a subject with Alzheimer's disease (AD) and cerebral amyloid angiopathy (CAA), retinal ganglion cell degeneration in glaucoma (RGCD), or myositis/myopathy (MM).

In certain embodiments, the composition comprises the ligand-bound zinc sulfide nanoparticles (R-ZnS NPs) as disclosed above and a pharmaceutically acceptable excipient. In certain embodiments, the excipient is phosphate-buffered solution, or physiological saline.

The present invention provides ligand-bound zinc sulfide nanoparticles (R-ZnS NPs) for use in treatment of a subject with Alzheimer's disease (AD) and cerebral amyloid angiopathy (CAA), retinal ganglion cell degeneration in glaucoma (RGCD) or myositis/myopathy (MM).

The present invention provides a use of the above disclosed R-ZnS NPs for treating a subject with Alzheimer's disease (AD) and cerebral amyloid angiopathy (CAA), retinal ganglion cell degeneration in glaucoma (RGCD) or myositis/myopathy (MM), or a method for treating a subject with Alzheimer's disease (AD) and cerebral amyloid angiopathy (CAA), retinal ganglion cell degeneration in glaucoma (RGCD) or myositis/myopathy (MM) using the above disclosed R-ZnS NPs. In certain embodiments, the method for treatment comprises administering a pharmaceutically effective amount of R-ZnS NPs to the subject. The pharmaceutically effective amount can be ascertained by routine in vivo studies. In certain embodiments, the pharmaceutically effective amount of R-ZnS NPs is a dosage of at least 0.001 mg/kg/day, 0.005 mg/kg/day, 0.01 mg/kg/day, 0.05 mg/kg/day, 0.1 mg/kg/day, 0.5 mg/kg/day, 1 mg/kg/day, 2 mg/kg/day, 3 mg/kg/day, 4 mg/kg/day, 5 mg/kg/day, 6 mg/kg/day, 7 mg/kg/day, 8 mg/kg/day, 9 mg/kg/day, 10 mg/kg/day, 15 mg/kg/day, 20 mg/kg/day, 30 mg/kg/day, 40 mg/kg/day, 50 mg/kg/day, 60 mg/kg/day, 70 mg/kg/day, 80 mg/kg/day, 100 mg/kg/day, 200 mg/kg/day, 300 mg/kg/day, 400 mg/kg/day, 500 mg/kg/day, 600 mg/kg/day, 700 mg/kg/day, 800 mg/kg/day, 900 mg/kg/day, or 1000 mg/kg/day.

In certain embodiments, the subject is human. In certain embodiments, the subject is a pet animal such as a dog.

The following examples are provided for the sole purpose of illustrating the principles of the present invention; they are by no means intended to limit the scope of the present invention.

### EMBODIMENTS

Currently, Aβ-induced cellular AD models and LPS-induced cellular inflammation models, as well as APP/PS1 double transgenic AD mouse models are widely used as experimental models.

### Embodiment 1

### Preparation of Captopril-bound Zinc Sulfide Nanoparticles (Cap-Zns NPs)

(1) dissolved the ligand captopril (43.46 mg) in deionized water (10 ml), resulting in a captopril aqueous solution; put the captopril aqueous solution into a reaction flask; then slowly added zinc acetate solution (0.01 mol/L, 10 mL) into the captopril aqueous solution, resulting in a captopril/zinc acetate reaction mixture; stirred the captopril/zinc acetate reaction mixture at room temperature for 0.5 hour; the molar ratio of captopril to zinc acetate in the captopril/zinc acetate reaction mixture was 2:1;
(2) added the newly prepared sodium hydroxide solution (1 M) to the captopril/zinc acetate reaction mixture to adjust the pH of the captopril/zinc acetate reaction mixture to 9, resulting in pH-adjusted captopril/zinc acetate reaction mixture, and stirred for 1 hour at room temperature.
(3) added slowly dropwise sodium sulfide aqueous solution (0.004 mol/L, about 10 ml) to the pH-adjusted captopril/zinc acetate reaction mixture, resulting in a sodium sulfide/captopril/zinc acetate reaction mixture; stirred the sodium sulfide/captopril/zinc acetate reaction mixture for 1 hour at room temperature; the molar ratio of sodium sulfide to zinc acetate in the sodium sulfide/captopril/zinc acetate reaction mixture is 0.4:1;
(4) moved the reaction flask with the sodium sulfide/captopril/zinc acetate reaction mixture into 60° C. oil bath and stirred for 2 hours, resulting in the formation of Cap-ZnS NPs;
(5) separated the formed Cap-ZnS NPs by centrifugation with an ultrafiltration tube with a molecular weight cut-off of 5k at 5000-6000 r/min for 5 minutes; and then ultrafiltration-washed the separated Cap-ZnS NPs three times with ultrapure water, resulting in purified Cap-ZnS NPs;
(6) freeze-dried the purified Cap-ZnS NPs, resulting in white stable Cap-ZnS NP powder.

### Embodiment 2

### Characterization of Cap-ZnS NPs

2.1. Particle sizes of Cap-ZnS NPs

At room temperature, Cap-ZnS NPs were suspended in a mixture of ethanol and water at a volume ratio of 1:1, and the particle size of Cap-ZnS NPs were measured by JEM-2100F transmission electron microscope (JEOL, Japan). 100 Cap-ZnS NPs were randomly counted by Image J to calculate the particles sizes.

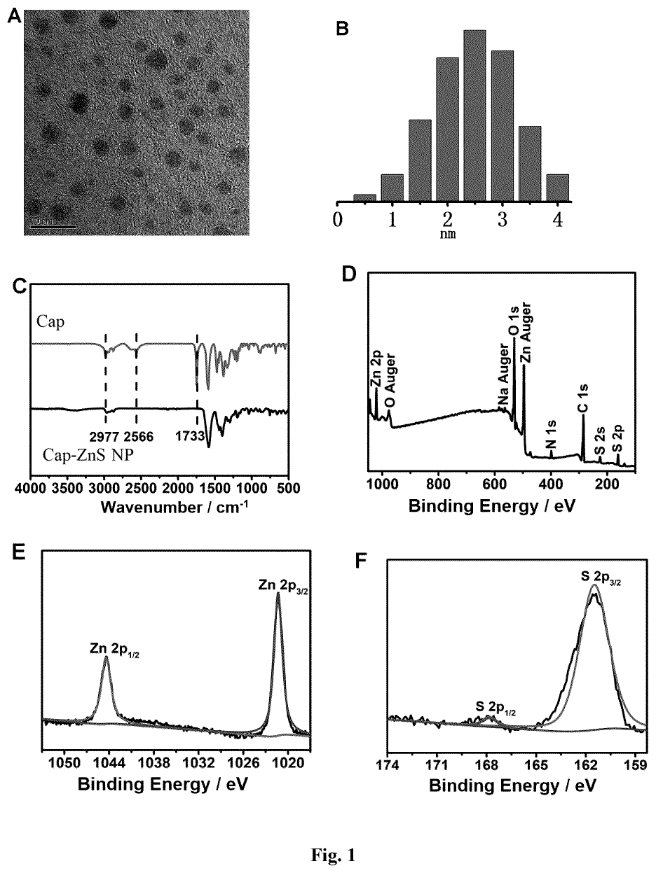

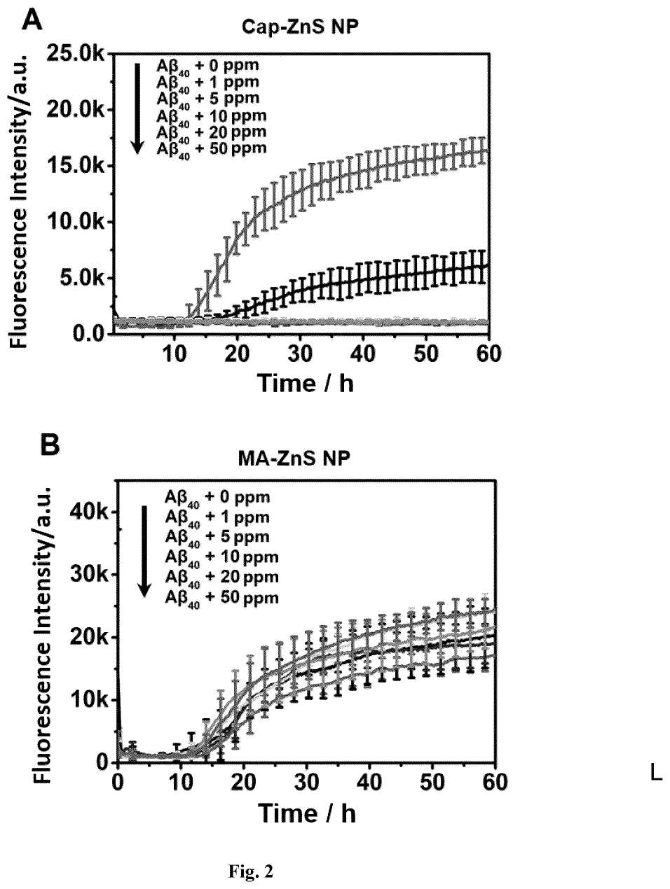

FIG. 1A is a representative transmission electron microscope photo, showing that the prepared Cap-ZnS NPs have a good dispersion. FIG. 1B shows the size distribution of Cap-ZnS NPs, which is mainly distributed in 0.5-4.0 nanometers (nm).

2.2. Infrared spectrum of Cap-ZnS NPs

At room temperature, the infrared spectra of Cap-ZnS NPs and Cap in the range of 4000-500 cm−1 were measured using the German Bruker vertex 80v FTIR. The freeze-dried samples were tested in MIR-ATR mode, and the results are shown in FIG. 1C.

The results showed that after the ligand Cap participated in the formation of Cap-ZnS NPs, the -SH stretching vibration characteristic peak (2566 cm−1) in the infrared spectrum disappeared, indicating that Cap was successfully grafted to the ZnS core through the Zn—S bond.

### 2.3. X-Ray Photoelectron Spectroscopy (XPS) of Cap-ZnS NPs

ESCALAB 250Xi XPS (Thermo Fisher, USA) was used to determine the elemental composition, content and binding energy of the total XPS spectra and single element spectra of C, N, O, S, and Zn. The single-element spectrum data were analyzed with XPS PEAK, and the results are shown in FIGS. 1D, 1E, and 1F.

The characteristic peaks of C1s, O1s, N1s, S2s, S2p and Zn2p in FIG. 1D, and the high-resolution XPS spectra of Zn2p in FIG. 1E) and S2p in FIG. 1F are very consistent with the theoretical predictions of Zn2p_{1/2}, Zn2p_{3/2}, S2p_{1/2} and S2p_{3/2}, indicating the existence of the ZnS core and the successful grafting of Cap (bound) to the surface of the ZnS core.

### Embodiment 3

### Anti-An Protein Fibrosis Ability of Cap-ZnS NPs and Its Comparison with Two Other Ligand-Bound Zinc Sulfide Nanoparticles

The other two ligand-bound zinc sulfide nanoparticles are 4-mercaptobutyric acid (MA)-bound zinc sulfide nanoparticles (MA-ZnS NPs) and dihydrolipoic acid (DHLA)-bound zinc sulfide nanoparticles (DHLA-ZnS NPs). Their preparation methods are similar to that of Cap-ZnS NPs, except that the ligand is replaced with MA and DHLA respectively. Their size ranges are also consistent with that of Cap-ZnS NPs.

### 3.1. Comparison of Anti-Protein Fibrosis Abilities of Cap-ZnS NPs, MA-ZnS NPs and DHLA-ZnS NPs by ThT Fluorescence Spectroscopy

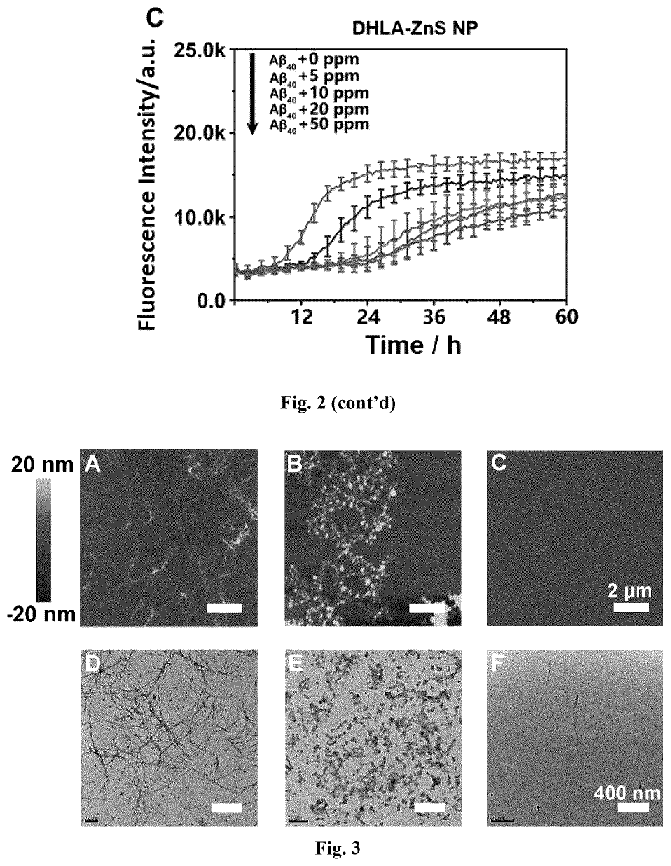

The kinetic process of Aβ40 fibrosis was studied using the Genetic Synergy™ MX microplate reader from Bio Tek, USA. The PBS buffer solution containing 40 μMAβ40 and 50 μM ThT was added to a 96-well plate with a black tube wall and a transparent glass bottom plate (final concentrations were 20 μM and 25 μM, respectively). The same volume of Cap-ZnS NPs, MA-ZnS NPs or DHLA-ZnS NPs samples with different concentrations was added to the final concentration of 0, 1, 5, 10, 20, 50 ppm respectively, and the plates with membrane seals were placed in the multi-function reading device (Synergy™ Multi-Mode MX), and the plate reading program was set up. Test conditions: scanning end-point fluorescence, excitation light wavelength is set to 445 nm, detection fluorescence emission wavelength is 485 nm. Keep the temperature at 37° C., shake the plate at a moderate intensity for 10 seconds at the end of every 10 minutes, and then measure the fluorescence emission intensity at 485 nm for continuous measurement for 60 hours. The fluorescence intensity of ThT was monitored to reflect the influence of three zinc sulfide nanoparticles bound by different ligands on the kinetics of Aβ40 fibrosis. The results are shown in FIG. 2.

FIG. 2A, FIG. 2B and FIG. 2C respectively show the effects of different concentrations of Cap-ZnS NPs, MA-ZnS NPs and DHLA-ZnS NPs on the fibrosis kinetics of Aβ40 at a concentration of 20 μM. The results show that Cap-ZnS NPs have excellent anti-Aβ protein fibrosis ability, completely inhibiting Aβ fibrosis at a concentration as low as of 5 ppm (the ThT fluorescence kinetic curve is completely flat). Although MA-ZnS NPs and DHLA-ZnS NPs also showed a certain ability to inhibit Aβ fibrosis, they still could not achieve complete inhibition of Aβ fibrosis at a concentration as high as of 50 ppm. Comparing the three ThT fluorescence kinetic curves, it can be seen that the inhibitory effect of Cap-ZnS NPs on Aβ fibrosis at 1 ppm has reached or exceeded that of the other two zinc sulfide nanoparticles at a final concentration of 50 ppm, indicating that the anti-protein fibrosis ability of Cap-ZnS NPs far exceeds the other two kinds of zinc sulfide nanoparticles.

### 3.2. Study the Effect of Cap-ZnS NPs on the Microscopic Morphology of Aβ40 Fibrosis by Atomic Force Microscopy (AFM)

At room temperature, a FastScan atomic force microscope (Bruker, Germany) was used to study the microscopic morphology of Aβ40 fibers after 60-hours incubation in the presence of different concentrations of Cap-ZnS NPs. It adopts ScanAsyst air mode, SNL-10 pin scan, and the image resolution is 512×512.

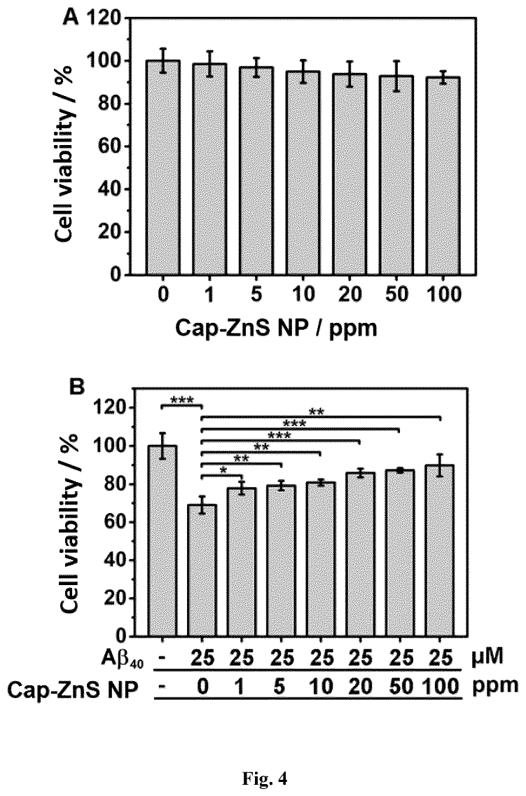

FIGS. 3A, 3B, and 3C show the results of the AFM test when the final concentration of Cap-ZnS NPs was 0, 1, and 5 ppm, respectively.

It can be seen that when Cap-ZnS NPs (0 ppm) was not present in the reaction system, a large number of Aβ fibers appeared; when the final concentration of Cap-ZnS NPs was 1 ppm, the fibrous structure of Aβ changed to aggregates composed of small rod-like original Fiber (proto-fibril); and when the final concentration of Cap-ZnS NPs reached 5 ppm, there was almost no Aβ fiber or fibril structure in the reaction system. These results are consistent with that of the fibrosis kinetics measured by the ThT fluorescence method. It further shows that 1 ppm Cap-ZnS NPs has shown a good Aβ fibrosis inhibitory effect, while 5 ppm Cap-ZnS NPs can achieve the effects of complete suppression.

### 3.3. Transmission Electron Microscope

A JEM-2100F transmission electron microscope (JEOL, Japan) was used to measure the morphological changes of Af340. The results are shown in FIGS. 3D, 3E, and 3F. The results are consistent with the AFM test results.

### Embodiment 4

### AD Model Test of Aβ-Induced PC-12 Cell Damages

PC-12 cells were obtained from Wuhan Procell Life Science & Technology Co., Ltd. CCK8 method was used to detect the cell viability of PC-12 cells. These cells were cultured in DMEM medium containing 10% FBS and 1% PS at a temperature of 37° C. and a CO2 concentration of 5%. After the cells have grown to a suitable number, 100 μL of cells in good condition were seeded in a 96-well plate with a density of 5×104 cells mL⁻¹ and cultured for 24 hours (n=6). Then, removed all the media co-incubated with the cells, and added different doses of Cap-ZnS NPs, Aβ40 or their mixture to a 96-well plate (100 μL per well), and incubated for another 22 hours. Subsequently, 100 μL of DMEM solution containing 10% CCK-8 was added to each well and incubated for 2 hours. The absorbance at 450 nm with a microplate reader was measured, and the results are shown in FIG. 4.

FIG. 4A shows the effect of different concentrations of Cap-ZnS NPs on the survival rate of PC-12 cells. It can be seen that when the final concentration of Cap-ZnS NPs reached 100 ppm, the cell survival rate still maintained above 92%, indicating that Cap-ZnS NPs have good safety at the cellular level. FIG. 4B shows the effect of different concentrations of Cap-ZnS NPs on the cell survival rate of the PC-12 cells in the presence of Aβ40 (final concentration of 25 μM). Aβ40 caused a significant decrease in the survival rate of PC-12 cells (from 100% to 68.5±5.2%), but the addition of Cap-ZnS NPs significantly restored the cell survival rate, and this effect increased significantly with the increase of the concentration of Cap-ZnS NPs. When the final concentration of Cap-ZnS NPs reached 100 ppm, the cell survival rate recovered to more than 90%. These results show that Cap-ZnS NPs can significantly reduce Aβ40-induced PC-12 cell damages, demonstrating the neuroprotective effect of Cap-ZnS NPs.

### Embodiment 5

### LPS-Induced Cell Inflammation Experiment

Test drugs: Cap-ZnS NPs, L-Cys-ZnS NPs, D-Cys-ZnS NPs, L-NIBC-ZnS NPs, D-NIBC-ZnS NPs, L-NAC-ZnS NPs, and D-NAC-ZnS NPs.

Human astroglia (HA) cells were obtained from Wuhan Procell Life Science & Technology Co., Ltd. The cell culture medium is DMEM medium containing 10% FBS and 1% PS. The culture temperature of the cell incubator is 37° C. and the CO2 concentration is 5%. HA cells in good condition were seeded in a 6-well plate at 2.4×108 cells/mL for culture. 7 groups: blank control group, LPS injury model control group, 4 test groups of Cap-ZnS NPs (final concentrations of 1 ppm, 5 ppm, 10 ppm or 20 ppm) (n=4) and 1 Cap control group (The final concentration of Cap was 20 ppm) (n=4). After the cells have grown for 24 hours, added DMEM minimal medium and Cap-ZnS NPs or Cap for pretreatment, and 2 hours later, added LPS (final concentration of 5 ppm). After culturing for another 24 hours, the culture medium and cells were collected, and the ELISA kit was used to detect the protein expression levels of inflammatory factors (IL-6, IL-8, IL-1(3, hs-CRP, TNF-α) in the cell culture medium. The specific methods are as follows: Taking 100 μL of the standard and sample diluent diluted to a specific concentration and adding it to a 96-well plate. After incubating for 90 minutes at 37° C., removing the liquid in the well, adding 100 μl of biotinylated antibody working solution and incubating for 1 hour, then washing the plate. After incubating with 100 μL of enzyme conjugate working solution for 0.5 h, washing the plate, incubating with 90 μL of chromogenic reagent (TMB) for 15 min in the dark, and stopping the reaction with 50 μL of stop solution. The microplate reader measures the absorbance at 450 nm.

The same experiments were performed using L-Cys-ZnS NPs, D-Cys-ZnS NPs, L-NIBC-ZnS NPs, D-NIBC-ZnS NPs, L-NAC-ZnS NPs, and D-NAC-ZnS NPs.

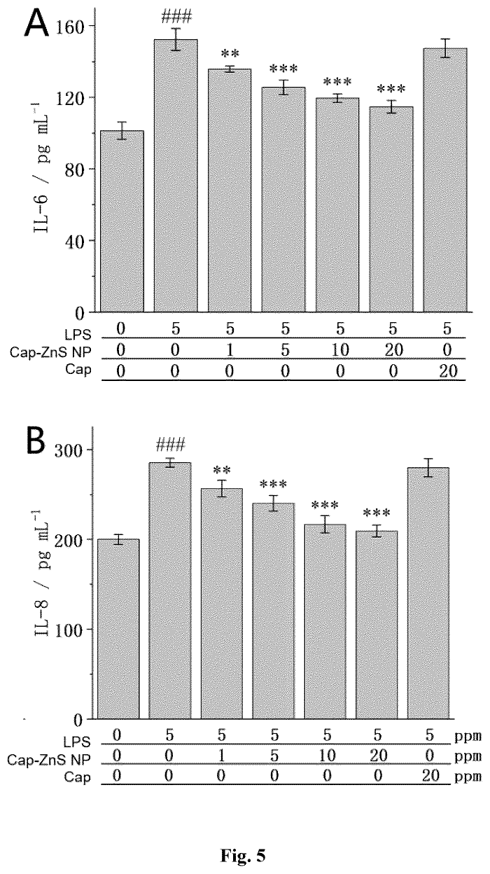

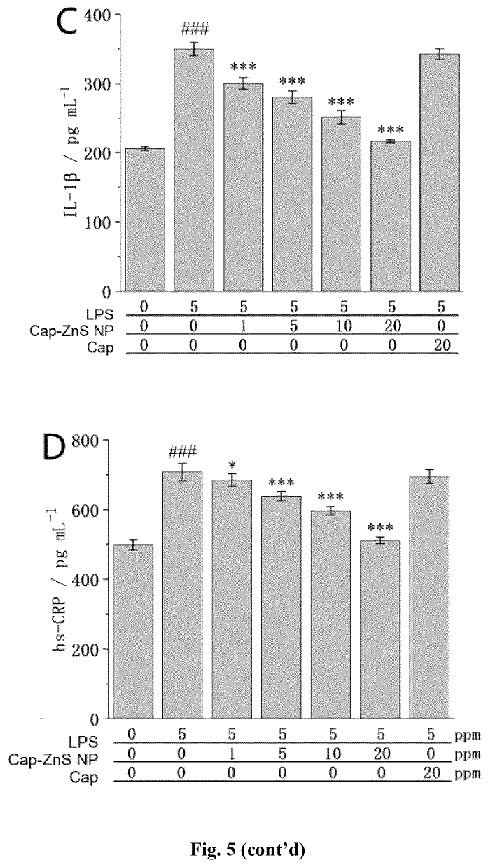

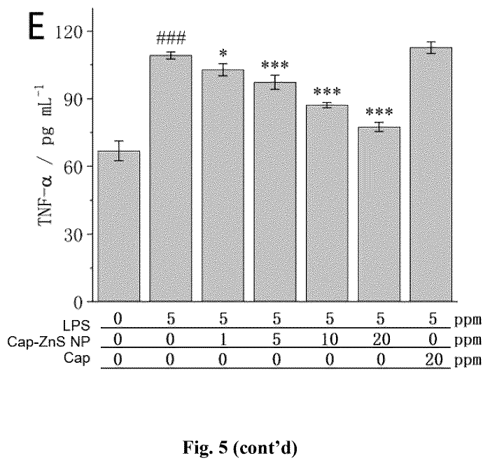

FIGS. 5A, 5B, 5C, 5D and 5E respectively show the protein expression levels of five inflammatory factors IL-6, IL-8, IL-1β, hs-CRP and TNF-α. It can be seen that LPS caused a substantial increase in five inflammatory factors (compared to the blank control group, P are all less than 0.001, ###), indicating that the model was successfully established. The addition of Cap-ZnS NPs significantly inhibits the increase of five inflammatory factors (compared to the LPS model control group, P are all less than 0.05, *, less than 0.01, **, or less than 0.001, ***), and with the increase of concentrations, this effect shows an obvious trend of enhancement. When the concentration of Cap-ZnS NPs reached 100 ppm, the levels of the five inflammatory factors almost dropped to levels similar to those in the normal control group. However, the levels of inflammatory factors in the Cap control group did not decrease significantly compared to the LPS model control group. The above results indicate that Cap-ZnS NPs exhibits excellent anti-inflammatory effects in cell experiments.

L-Cys-ZnS NPs, D-Cys-ZnS NPs, L-NIBC-ZnS NPs, D-NIBC-ZnS NPs, L-NAC-ZnS NPs, and D-NAC-ZnS NPs also showed outstanding anti-inflammatory effects, similar to Cap-ZnS NPs; for the brevity, detailed descriptions will be omitted.

### Embodiment 6

### Acute Toxicity, and Tissue Distribution and Metabolism Tests in Mice

6.1. Test method
(1) Maintenance of Mice

Forty-two clean Kunming mice, 6-8 weeks old, weighing 25-30 kg, 21 male and female mice each, were housed in ordinary cages with 12 hours of light and darkness each day. Mice had free access to food and water. Male and female mice were randomly selected and divided into 1-6 groups (7 mice/group) for experimentation.
(2) Tissue Treatment

Group 1 and Group 2 were used for acute toxicity test in mice. The Group 1 was the drug test group, and the Group 2 was the blank control group. The drug test group was injected with 100 mg/Kg mouse body weight of Cap-ZnS NPs drug by intraperitoneal injection, and the blank control group was injected with the same volume of normal saline. The mice were sacrificed 24 hours later. After perfusion with normal saline, the heart, liver, spleen, lung, kidney, and brain were dissected out and fixed in 4% paraformaldehyde. Put the fixed animal tissue into the embedding box, and rinsed with running water for 30 minutes to remove the paraformaldehyde in the tissues. The tissues were dehydrated with an alcohol gradient to be transparent in xylene. The transparent tissues were immersed in a 1:1 mixture of paraffin wax and xylene for 90 minutes, then placed in paraffin wax for 2 hours, and immediately cooled. Used a paraffin microtome to make 51.tm continuous slices of the tissues, and baked slices at 60° C. for 2 hours. Immersed the slices in xylene for 5 minutes to deparaffinize, repeated 3 times. Then the slices were immersed in gradient ethanol (100%, 90%, 80%, and 70%) for 5 minutes each, and rinsed with running tap water for 5 minutes. Stained the section in hematoxylin staining solution for 5 minutes, then washed the excess staining solution on the slide with tap water, separated the color with 0.7% hydrochloric acid and ethanol for 10 seconds, rinsed the slide with tap water until the nucleus and nuclear chromatin are clear under the microscope. After dehydration with 70% and 90% ethanol for 10 minutes, dyed with 0.5% eosin solution for 5 minutes, and rinsed the excess dye solution with running water. The stained sections were dehydrated with 70%, 80%, 90%, and 100% ethanol for 10 seconds, soaked in xylene for 1 minute to make the tissue transparent, and air-dried naturally in a ventilated place. Quickly added proper amount of neutral gum and mount the slides. The pathological slices were observed and photographed with an optical microscope, and 2 fields of view were randomly selected for each slice to analyze all the tissues.

Groups 3 to 6 were used to explore the tissue distribution of drugs. Each group was administered Cap-ZnS NPs by intraperitoneal injection at the amount of 20 mg/Kg mouse body weight, and sacrificed after 2, 6, 12, or 24 hours. After dissection, the heart, liver, spleen, lung, kidney and brain were immediately put into liquid nitrogen and freeze-dried. After five days, it was taken out and ground into a uniform powder. Weighed 2 mg of tissue powder and digested it in a mixed solution of concentrated nitric acid and hydrogen peroxide (volume ratio 5:1) for inductive coupling Plasma emission spectroscopy tests the content of Cap-ZnS NPs in the heart, liver, spleen, lung, kidney and brain.

### 6.2. Test Results

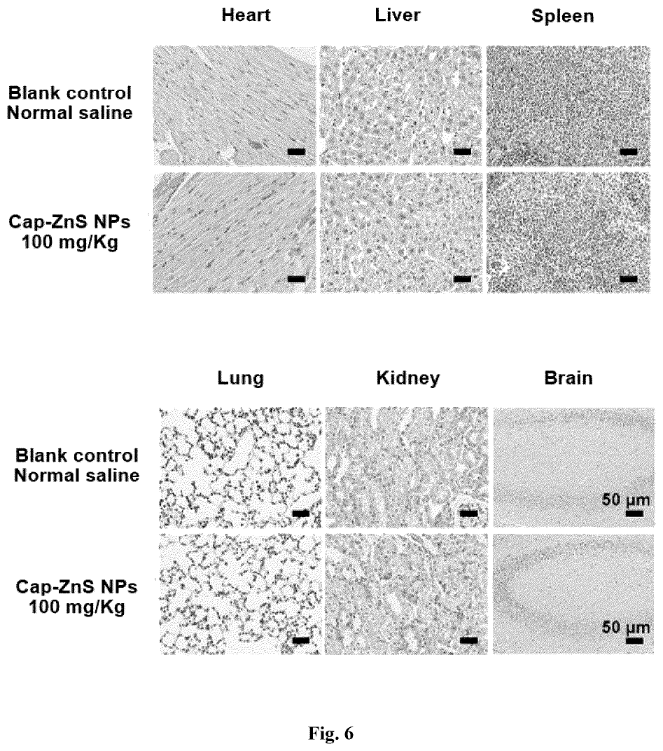

Acute toxicity studies found that Cap-ZnS NPs have no effect on the eyes, skin and mucous membranes of mice, as well as the breathing, food intake, exercise and excretion of mice within 24 hours. Further pathological examination found that, as shown in FIG. 6, compared with the blank control group (top), the main organs of the mice in the Cap -ZnS NPs test group, including the heart, liver, spleen, lung, and brain tissue, were arranged normally, and no infiltration of inflammatory cells was found. The above research shows that Cap-ZnS NPs do not cause obvious toxic and side effects to normal tissues and organs, and have good biological safety.

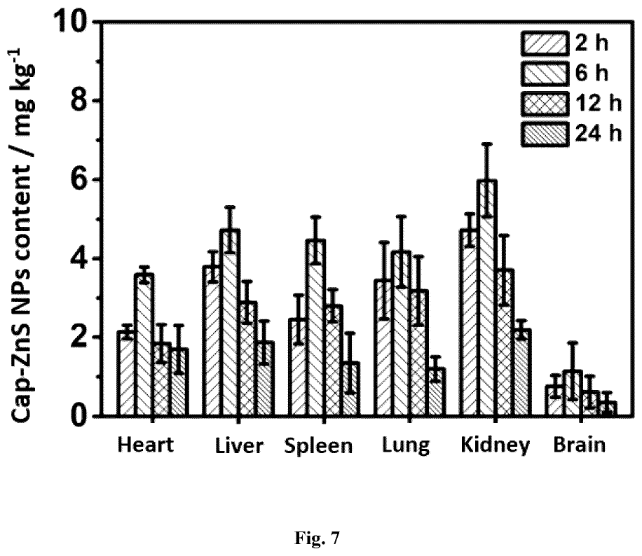

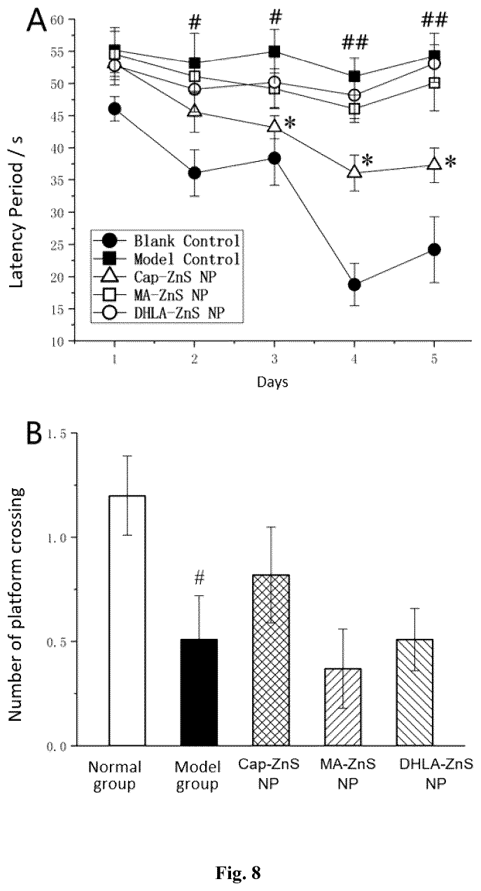

FIG. 7 shows the content of Cap-ZnS NPs in heart, liver, spleen, lung, kidney and brain tissues. The results showed that the content of the drug in each organ reached the maximum in about 6 hours, and then gradually decreased with time. A considerable amount of drugs was also observed in the brain, indicating that the drugs can penetrate the blood-brain barrier and enter the brain.

### Embodiment 7

### Test with APP/PS1 Double Transgenic AD Model Mice

### 7.1. Test Methods

Test drugs: Cap-ZnS NPs, L-Cys-ZnS NPs, D-Cys-ZnS NPs, L-NIBC-ZnS NPs, D-NIBC-ZnS NPs, L-NAC-ZnS NPs, D-NAC-ZnS NPs, MA-ZnS NP and DHLA-ZnS NP.

The test mice were 60-week-old C57BL/6 germline APP/PS1 transgenic AD model mice. The model mice were randomly divided into model control group, Cap-ZnS NPs administration group, L-Cys-ZnS NPs administration group, D-Cys-ZnS NPs administration group, L-NIBC-ZnS NPs administration group, D-NIBC-ZnS NPs administration group, L-NAC-ZnS NPs administration group, D-NAC-ZnS NPs administration group, MA-ZnS NPs administration group and DHLA-ZnS NPs administration group. At the same time, C57BL/6 wild-type mice group with the same age was set as a normal control group. 15 mice in each group. Each administration group was intraperitoneally injected with physiological saline solution of the corresponding drug once a day, the dose was 20 mg/Kg mouse body weight, and the injection volume was 100 μL. The mice of the model control group and normal control group were intraperitoneally injected with the same volume of physiological saline .

After 4 weeks of continuous administration, the Morris water maze test was used to analyze the cognitive and memory functions of all animals.

Place navigation test: The Morris water maze test system is composed of a water maze and an automatic video recording and analysis system. A camera is disposed above the water maze and connected to a computer. The water maze consists of a circular pool with a diameter of 120 cm and a height of 60 cm, and a platform with a diameter of 9 cm. The liquid level is 0.5 cm higher than the platform, and the water temperature is maintained at 22±0.5° C. Use white pigment to dye water to milky white. The place navigation test was used to measure the learning and memory abilities of mice in the water maze, which lasted 5 days. The water maze is divided into four quadrants: N, S, W, E. The platform is placed in a fixed quadrant. The position of the platform is fixed throughout the experiment. During training, the mice with head towards the pool wall were gently put into the water close to the outer wall from the ½ arc of different quadrants every day. Record the time when the mice climb on the hidden platform or stop the test when the time reaches 60 s. After the mice are on the platform, let them stay on the platform for 30 s. If the mice do not find the platform within 60 s, the experimenter guides the mouse to climb on the platform and let it stay for 30 s. The latency period in which mice seek the platform during the test was recorded by the camera tracking system. After the test is over, each animal is removed and dried gently with a hair dryer. Each animal was trained 4 times a day, with an interval of 20 minutes between trainings, for 5 consecutive days.

Space exploration test: after completing the training on the 5th day, the platform was removed on the 6th day, and the mice were gently placed in the water from the most distal point of the platform while facing the wall of the pool. Each mouse's movement trajectory within 60 seconds was recorded by the camera, and analyzed by software were the number of times the mouse crossed the platform, the stay time in the target quadrant, and the swimming time in the target quadrant.

The test results are expressed as ,C±SEM, all data are processed by SPSS software (SPSS 21), and a one-way analysis of variance (Post-Hoc Dunnett test) is used; P<0.05 indicates that the difference is statistically significant. Kruskal-Wallis H test and Mann-Whitney U test are used for statistical analysis of data that are not normally distributed.

After the behavioral tests, the mice were anesthetized by intraperitoneal injection of 7% chloral hydrate, and the cardiac perfusion connection was established, followed by rapid flushing with normal saline for 7 minutes, and then used 4% chloral hydrate to fix the tissues for 7 minutes. After the perfusion was over, the brain tissue was carefully taken and placed in 4% perfusate, and stored at room temperature for later use. Immunohistochemical methods were used to detect the expression of Aβ40 and inflammatory factors in the hippocampus and cortex: the perfused tissue was dehydrated, made transparent, waxed and embedded, and then sectioned using a paraffin microtome. A gradient of xylene and absolute ethanol was used for dewaxing. After antigen retrieval by microwave, the slices were incubated with hydrogen oxide, and the slices were blocked with serum for 30 min. Added primary antibodies against Aβ40, IL-10, TNF-α, GFAP, IL-6, or COX-2 (1:100) at room temperature, and incubated overnight (15 h) at 4° C. Discarded the primary antibody, washed the slices with PBS, ed HRP-labeled goat anti-rabbit/mouse secondary antibody, and incubated at room temperature for 30 min. After rinsing the slices with PBS, added the chromogenic reagent DAB chromogenic solution to develop the color, Harris hematoxylin counterstaining, dehydration, and mounting. A fluorescence microscope was used to take pictures, and Image J was used to quantitatively analyze the slices.

### 2. Test Results

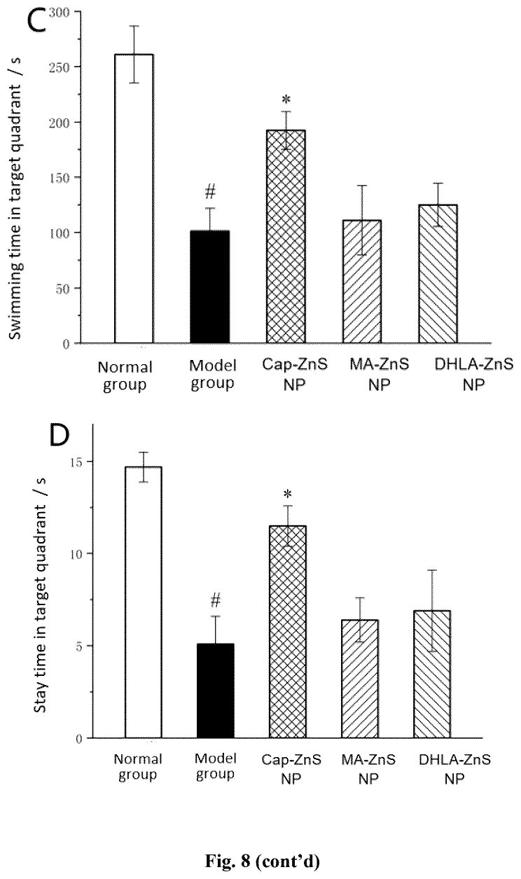

FIG. 8 shows the effects of Cap-ZnS NPs, MA-ZnS NPs and DHLA-ZnS NPs on the performance of male mice in the Morris water maze after being given daily for 4 consecutive weeks. During the training process of the place navigation test (FIG. 8A), compared with the normal control group (●), from the 2nd day to the 5th day of training, the latency period of the model control group (▪) mice was significantly higher than the normal mice (P<0.05, #; P<0.01, ##). Compared with the model control group, the Cap-ZnS NPs administration group (Δ) can greatly reduce the latency period of mice, and there are significant differences from the model control group from the 3rd day onwards (The P values in 3rd, 4th, 5 days were all less than 0.05, *). However, MA-ZnS NPs (□) and DHLA-ZnS NPs (◯) administration groups had no significant effect on reducing the latency period.

The results of the space exploration test (FIG. 8B-D) showed that compared with the normal control group, the model control group had significant decreases of the number of platform crossings (FIG. 8B) (P<0.05, #), the swimming time in the target quadrant (FIG. 8C) (P<0.05, #), and the stay time in the target quadrant (FIG. 8D) (P<0.05, #). Compared with the model control group, MA-ZnS NPs and DHLA-ZnS NPs failed to increase the number of platform crossings in mice (FIG. 8B), while the number of platform crossings in the Cap-ZnS NPs administration group increased apparently, but there was no statistical difference (P>0.05).

For the target quadrant swimming time and target quadrant stay time (FIG. 8C and FIG. 8D), Cap-ZnS NPs drugs significantly increased these two values (P less than 0.05, *), but both MA-ZnS NPs and DHLA-ZnS NPs failed to increase these two values.

The above data show that Cap-ZnS NPs drugs can significantly improve the cognitive and memory abilities of AD model mice, while MA-ZnS NPs and DHLA-ZnS NPs have no such effect.

The effects of L-Cys-ZnS NPs administration group, D-Cys-ZnS NPs administration group, L-NIBC-ZnS NPs administration group, D-NIBC-ZnS NPs administration group, L-NAC-ZnS NPs administration group and D-NAC-ZnS NPs administration group are similar to that of the Cap-ZnS NPs administration group. For the sake of brevity, they will not be described in details herein.

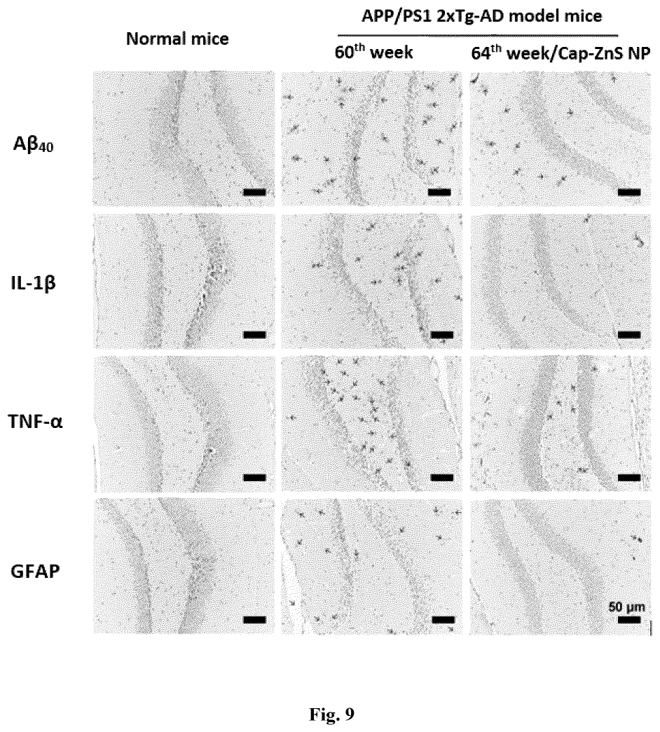

FIG. 9 shows representative photos of the immunohistochemistry results. As shown in FIG. 9, the hippocampus of wild-type mice does not show Aβ40 plaques or express prominent inflammatory factors including IL-1β, TNF-α and GFAP. Compared with the wild-type mice in the normal control group, there are a large number of Aβ40 plaques and the expressions of IL-1β, TNF-α and GFAP in the hippocampus of the model control group. The hippocampus of Cap-ZnS NPs administration group shows only a small amount of Aβ40 plaques and insignificant expression of TNF-α, IL-1β and GFAP, which were close to the normal control group. The statistical results showed that the number of Aβ40 plaques in the hippocampus of the Cap-ZnS NPs administration group decreased by 71.8% compared with the model control group, with significant statistical difference (P<0.01), and the expression of TNF-α, IL-1β and GFAP decreased exceeding 70% (P<0.01). The above studies show that the administration of Cap-ZnS NPs can significantly reduce the Aβ plaques in the brain of AD model mice and greatly inhibit the central inflammation in AD model mice, thereby reducing neuronal cell damage, and then curing AD. Because the AD model mice used in these tests are 60-week-old old mice, and the administration cycle is only 4 weeks, such a good effect has been achieved, indicating that Cap-ZnS NPs drugs have excellent applications in the treatment of AD.

The R-ZnS NPs described in the present invention have the characteristics of simple preparation method and good biocompatibility.

R-ZnS NPs provided by the present invention have the following advantages:

First, R-ZnS NPs are significantly more effective in inhibiting Aβ fibrosis than other ligand (such as 4-Mercaptobutyric Acid (MA)- and dihydrolipoic acid, DHLA)-bound ZnS NPs; and R-ZnS-NPs at the ultra-low dose of 5 ppm can completely inhibit the fibrosis of Aβ at a concentration of 20 μM.

Second, R-ZnS NPs can greatly reduce the expression of pro-inflammatory factors (IL-1β, IL-6, TNF-α, IL-8 and hs-CRP) in LPS-treated HA cells.

Third, R-ZnS NPs significantly reduced the cytotoxicity caused by Aβ fibrosis in the Aβ-induced cell damage model experiment.

Fourth, R-ZnS NPs significantly reduced Aβ plaques in the hippocampus of AD model mice in the APP/PS1 double transgenic AD mouse model tests, and significantly reduced the level of neuroinflammatory factors in the brain of AD model mice. In the APP/PS1 double transgenic AD model mouse tests, R-ZnS NPs significantly improved the cognitive and memory behavioral obstacles of the model mice.

Fifth, R-ZnS NPs can penetrate the blood-brain barrier and enter the mouse brain.

Sixth, R-ZnS NPs have biological safety at the animal level.

The ligand-bound zinc sulfide nanoparticles with ligands of L-cysteine, D-cysteine, L-NIBC, D-NIBC), L-NAC or D-NAC have been synthesized, characterized, and tested following the same protocols as described above, and have shown the similar effects on reducing the expression of inflammatory cytokines, inhibiting Aβ fibrillation, and treating Aβ-related diseases such as AD. For the sake of brevity, they will not be described in details herein.

While the present invention has been described with reference to particular embodiments, it will be understood that the embodiments are illustrative and that the invention scope is not so limited. Alternative embodiments of the present invention will become apparent to those having ordinary skill in the art to which the present invention pertains. Such alternate embodiments are considered to be encompassed within the scope of the present invention. Accordingly, the scope of the present invention is defined by the appended claims and is supported by the foregoing description.

1. A ligand-bound zinc sulfide nanoparticle, comprising:
a zinc sulfide core; and
a ligand bound to the zinc sulfide core.

2. The ligand-bound zinc sulfide nanoparticle of claim 1, wherein the diameter of the zinc sulfide core is 0.5-4.0 nm.

3. The ligand-bound zinc sulfide nanoparticle of claim 1, wherein the diameter of the zinc sulfide core is 1.0-3.5 nm.

4. The ligand-bound zinc sulfide nanoparticles of claim 1, wherein the ligand is one selected from the group consisting of L-cysteine, D-cysteine, N-isobutyryl-L-cysteine (L-NIBC), N-isobutyrul-D-cysteine (D-NIBC), N-ace 1-L-cysteine (L-NAC), N-acetyl-D-cysteine (D-NAC), and Captopril.

5. (canceled)

6. A process of preparing ligand-bound zinc sulfide nanoparticles (R-ZS NPs), said process comprising:
dissolving a ligand in deionized water, resulting in a ligand aqueous solution;
wherein the concentration of ligand in the ligand aqueous solution is 0.02-2.0 mol/L;
adding zinc acetate solution into the ligand aqueous solution, resulting in a zinc acetate/ligand reaction mixture; wherein the concentration of the zinc acetate aqueous solution is 0.01-1.0 mo/L; and wherein the molar ratio of ligand to zinc acetate ranges from 1:1 to 10:1;
adjusting the pH of the zinc acetate/ligand reaction mixture to the range of 7-10;
adding sodium sulfide aqueous solution dropwise into the pH-adjusted zinc acetate/ligand reaction mixture, resulting in a sodium sulfide/zinc acetateiligand reaction mixture; wherein the molar ratio of the added sodium sulfide to the zinc acetate in the zinc acetate/ligand reaction mixture ranges from 0.1:1-5:1;
heating the sodium sulfide/zinc acetate,/ligand reaction mixture to a predetermined temperature, and the reaction is maintained for a predetermined time, resulting in the formation of R-ZnS NPs; wherein the predetermined temperature is 50-100 degrees Celsius; and wherein the predetermined time is 1-5 hours.

7. The process of claim 6, wherein the process further comprises:
purifying the R-ZnS NPs by centrifugation with an ultrafiltration tube; wherein the ultrafiltration tube is with a molecular weight cut-off of 5 k Daltons.

8. (canceled)

9. (canceled)

10. (canceled)

11. (canceled)

12. A method for treating a disease or a condition of excessive expression of a cytokine in a subject, wherein the method comprises:
administering a composition to the subject with the disease or condition of excessive expression of a cytokine;
wherein the composition comprises a ligand-bound zinc sulfide nanoparticle; and
a pharmaceutically acceptable excipient;
wherein the ligand-bound zinc sulfide nanoparticle comprises:
a zinc sulfide core; and
a ligand bound to the zinc sulfide core; and
wherein the disease is selected from the group consisting of Alzheimer's disease (AD) and cerebral amyloid angiopathy (CAA), retinal ganglion cell degeneration in glaucoma (RGCD) and myositis/myopathy (MM); and
wherein the cytokine is selected from the group consisting of interleukin-6 (IL-6), interleukin-8 (IL-8), interleukin-1β (IL-1β), hypersensitive-c-reactive-protein (Hs CRP), and tumor necrosis factor-alpha (TNFα).

13. The method of claim 12, wherein the diameter of the zinc sulfide core is 0.5-4.0 nm.

14. The method of claim 12, wherein the diameter of the zinc sulfide core is 1.0-3.5 nm.

15. The method of claim 12, wherein the ligand is one selected from the group consisting of L-cysteine, D-cysteine, N-isobutyryl-L-cysteine (L-NIBC), N-isobutynil-D-cysteine (D-NIBC), N-acetyl-L-cysteine (L-NAC), N-acetyl-D-cysteine (D-NAC), and Captopril.

16. A composition for treatment of a disease or a condition of excessive expression of a cytokine in a subject, wherein the composition comprises a ligand-bound zinc sulfide nanoparticle; and a pharmaceutically acceptable excipient;
wherein the ligand-bound zinc sulfide nanoparticle comprises:
a zinc sulfide core; and
a ligand bound to the zinc sulfide core; and
wherein the disease is selected from the group consisting of Alzheimer's disease (AD) and cerebral amyloid angiopathy (CAA), retinal ganglion cell degeneration in glaucoma (RGCD) and myositis/myopathy (MM); and
wherein the cytokine is selected from the group consisting of interleukin-6 interleukin-8 (IL-8), interleukin-β (IL-1β), hypersensitive-c-reactive-protein (Hs CRP), and tumor necrosis factor-alpha (TNFα).

17. The composition of claim 16, wherein the diameter of the zinc sulfide core is 0.5-4.0 nm.

18. The composition of claim 16, wherein the diameter of the zinc sulfide core is 1.0-3.5 nm.

19. The composition of claim 16, wherein the ligand is one selected from the group consisting of L-cysteine, D-cysteine, N-isobutyryl-L-cysteine (L-NIBC), N-isobutyrul-D-cysteine (D-NIBC), N-acetyl-L-cysteine (L-NAC), N-acetyl-D-cysteine (D-NAC), and Captopril.
